(19) United States
(12) Patent Application Publication
(10) Pub. No.: US 2003/0106605 A1
(43) Pub. Date: Jun. 12, 2003

(54) Material having one or more chemistries which produce topography, unique fluid handling properties and/or bonding properties thereon and/or therein

(76) Inventors: Lee Kirby Jameson, Roswell, GA (US); Teresa De Jesus Petryk, Woodstock, GA (US); Varunesh Sharma, Atlanta, GA (US); Phillip A. Schorr, Atlanta, GA (US)

Correspondence Address: William W. Letson, Kimberly-Clark Worldwide, Inc., Patent Department, 401 North Lake Street, Neenah, WI, 54956, US

(21) Appl. No.: 09/990,686

(22) Filed: Nov. 16, 2001

(51) Int. Cl. A61F 13/20; A61F 13/15; B65B 3/04; B67C 3/02; B65B 1/04

(52) U.S. Cl. 141/98; 604/358

(57) ABSTRACT

The present invention relates a material having fluid handling characteristics and generally includes a substrate having at least a first surface, and one or more chemistries, being applied to the first surface of the substrate in discrete droplets, wherein one or more droplets form discrete domains on the substrate. The chemistries applied to the substrate may penetrate the substrate to varying degrees wherein the varying degrees of chemistry penetration produce materials having various topographies. The topography or topographies may create fluid barriers, skin separation, or otherwise result in skin health benefits. A second embodiment of the present invention is directed to an absorbent article having one or more phase-change materials applied thereto. A third embodiment is directed to a composite including a substrate and one or more chemistries, wherein the chemistries are applied to the substrate so as to produce a substrate having discretely placed and registered bond points.

### BACKGROUND OF THE INVENTION

Drop on demand valved ink jet printing apparatus have been used to apply inks to a variety of substrates for a period of time. Generally, a drop on demand valved ink jet printing apparatus operates to discharge individual droplets of ink onto a substrate in a predetermined pattern to be printed. Such an apparatus typically incorporates an array of orifices in a nozzle block, a plurality of control valves, and a controller. The orifices are customarily arranged in a vertical row, and conventional ink jet printing apparatus have incorporated a separate valve communicating with each orifice. The valves are controlled by the controller, which can be keyed by an operator to open and close the nozzles according to a programmed schedule to print one or a series of characters or symbols.

Each orifice is designed to emit a single droplet of ink during each opening of its associated valve. The droplets, emitted according to the programmed sequence, are directed toward a substrate where the character or symbol is printed. The quality of print produced by a drop on demand ink jet printer requires among other things, precise control over the size of the ink dot that impacts the substrate. Dot size in turn is affected by the size of an ink droplet discharged from a nozzle.

In the past, it was important in the overall design represented by the relationship between valve characteristics, orifice size, and ink characteristics, that the droplets not only be of proper size but also that the size be consistent because otherwise the printed characters or symbols would be irregular in width.

Typically, in early ink jet printing apparatus, a nozzle orifice array consisted of a vertical row of seven orifices coupled with seven control valves. Each control valve controlled the flow of ink through its associated orifice. An example of such a drop on demand ink jet printing apparatus is described and illustrated in U.S. Pat. No. 4,378,564. The subject matter disclosed by that patent is incorporated herein by reference.

In time, the need developed for an increased number of orifices. To meet this need, a larger number of orifices were assembled in a taller vertical array, and a correspondingly greater number of valves were incorporated, again, each nozzle orifice having its own control valve. The typical approach was to increase the number of orifices by superimposing two or more orifice nozzle arrays, each array incorporating the same number of valves as orifices. Of course, with each increase in the number of valves, the cost of the printing apparatus also increased.

In much of the ink jet printing done heretofore, the spacing between orifices has produced a printed character or symbol composed of essentially discrete dots of ink. Because of the number of them, these discrete dots have been acceptable in producing a readable character or symbol. However, the traditional ink jet printing apparatus was not acceptable to print bar codes because of the specifications for bar code printing required to assure accurate reading of the bar codes. Heretofore, nor have traditional ink jet printing apparatus been acceptable for the printing of non-ink chemistries where continuity of chemistry application, at least in certain regions, may be important.

While many improvements to conventional ink jet printing apparatus have been made, the ink jet printing apparatus currently available lack the ability to process phase-change liquids or materials and/or the ability to provide enhanced fluid handling characteristics (e.g. topography or fluid barrier) in a single pass of the apparatus across the substrate (or a single pass of the substrate past the apparatus). Additionally, due to the separation of discrete segments upon application to the substrate, conventional ink jet printing apparatus are not acceptable for the printing of non-ink chemistries where continuity of chemistry application, at least in certain regions, may be important.

### SUMMARY OF THE INVENTION

The present invention relates to a material having enhanced fluid handling characteristics. The material includes (i) a substrate, said substrate having at least a first surface; and (ii) one or more chemistries applied to the first surface of the substrate in discrete droplets; wherein one or more droplets form discrete domains on the substrate and wherein the domains produce a topography of chemistries upon the substrate. In one embodiment of the material of the present invention the chemistries applied to the substrate may have varying degrees of penetration into the substrate, such that the varying degrees of chemistry penetration results in a material having various topographies. In another aspect of the invention the one or more chemistries may be selectively applied to all or a portion of the substrate. The chemistries may be applied to the substrate in a pattern which is repeating, non-repeating or random and may be applied to the substrate so as to produce a fluid barrier. The chemistries applied to the material to produce a fluid barrier may be contiguously or discretely placed droplets or segments.

In yet another alternative embodiment, the present invention is also directed to an absorbent article comprising (i) a substrate, the substrate having at least a first surface; and (ii) one or more chemistries, where at least one of the chemistries, is at least in part, a phase-change liquid; and wherein the one or more chemistries are applied to the substrate in one application so as to produce a substrate having desired topographical characteristics. The one or more chemistries may be selectively applied to all or a portion of the substrate of the absorbent article, may be applied to the substrate in a pattern and/or may be applied to the substrate so as to create a topography of chemistry. Where a topography of chemistry is created on the substrate, the substrate will desirably exhibit skin health benefits such as improved fluid management (e.g. liquid wicking or flow management applications), and/or skin separation.

Another embodiment of the present invention is directed to a composite including a substrate, and one or more chemistries, wherein the substrate has at least one layer and has at least a first surface, and wherein at least one of the chemistries is, at least in part, a phase-change liquid. In this embodiment, the one or more chemistries are applied to the substrate so as to produce a substrate having discretely placed and registered bond points. The discrete bond points may be inter-fiber bond points or interfacial bond points.

### BRIEF DESCRIPTION OF THE DRAWINGS

FIG. 1 is an enlarged illustration of an exemplary configuration of a representative material of the present invention, wherein the material has a chemistry or application applied to the top surface of the material.

FIG. 2 is a cross-sectional view of a material representative of the present invention.

FIG. 3 is a enlarged cross-sectional view of a material representative of the present invention.

### DEFINITIONS

As used herein the following terms have the specified meanings, unless the context demands a different meaning, or a different meaning is expressed; also, the singular generally includes the plural, and the plural generally includes the singular unless otherwise indicated.

As used herein, the terms “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof.

As used herein, the term “fabric” refers to all of the woven, knitted and nonwoven fibrous webs.

As used herein, the term “layer” when used in the singular can have the dual meaning of a single element or a plurality of elements.

As used herein, the terms “lotion” or “ointment” are generally interchangeable and mean a formulation, powder or combination thereof comprising skin health ingredients, or compositions which are skin compatible but which do not in and of themselves provide skin health or skin wellness benefits.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein “multi-layer laminate” means a laminate wherein some of the layers are spunbond and some meltblown such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multi-layer laminates may also have various numbers of meltblown layers or multiple spunbond layers in many different configurations and may include other materials like films (F) or coform materials, e.g. SMMS, SM, SFS, etc.

As used herein the terms “nonwoven” and “nonwoven fabric or web” mean a web having a structure of individual fibers, filaments or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “personal care product” or “personal care absorbent product” means diapers, training pants, swim wear, absorbent underpants, baby wipes, adult incontinence products, sanitary wipes, wet wipes, feminine hygiene products, wound dressings, nursing pads, time release patches, bandages, mortuary products, veterinary products, hygiene and absorbent products.

As used herein, the term “petrolatum” refers to a semisolid mixture of hydrocarbons obtained from petroleum, such as, but not limited to Glenpure L White Petrolatum, USP available from Glenn Corporation, a business having offices in St. Paul, Minn.

As used herein, the term “phase-change” application, chemistry, liquid, material or the like refers to a material which is processed in a liquid or substantially liquid state and then solidifies or returns to its natural state when cooled.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

As used herein, the term “topical application” means any overlayer type of material surface modification, including, but not limited to any polishes, cleaning or cleansing agents, and the like, as well as any lotions, ointments, powders or the like and combinations thereof. For purposes of this application, the term “surface enhancing agent” is generally interchangeable with the term topical application.

These terms may be defined with additional language in the remaining portions of the specification.

### DETAILED DESCRIPTION OF THE INVENTION

### DESCRIPTION OF THE INVENTION

One aspect of the present invention relates to a material having one or more chemistries or applications discretely placed and registered thereon to provide a material which exhibits enhanced fluid handling characteristics. The material includes (i) a substrate, said substrate having at least a first surface; and (ii) one or more chemistries applied to the first surface of the substrate in discrete droplets; wherein one or more discrete and registeredly placed droplets form discrete domains on the substrate and wherein the domains produce a topography of chemistries upon the substrate. In one embodiment of the material of the present invention the chemistries applied to the substrate may have varying degrees of penetration into the substrate, such that the varying degrees of chemistry penetration results in a material having various topographies. In another aspect of the invention the one or more chemistries may be selectively applied to all or a portion of the substrate. The chemistries may be applied to the substrate in a pattern which is repeating, non-repeating or random and may be applied to the substrate so as to produce a fluid barrier. The chemistries may be applied such that the discrete segments thereof are overlapping or contiguously placed, and/or, in some instances, interconnected (i.e. formed of discrete droplets which merge or combine) to form discrete domains or regions. The contiguously placed segments may produce or create areas or domains of the substrate having, for example, fluid barrier properties, or channeling characteristics, etc. Another aspect of the material of the present invention is that the topography of chemistries may provide for improved fluid management and/or skin separation.

It is also contemplated that the topography of chemistry may be selected from topographies which are releasable or permanent. That is, the chemistries may release from the substrate or other chemistry when exposed to certain conditions or upon the happening of certain events (e.g. exposure to certain temperatures (e.g. at least about body temperature (about 23° C.), insult, etc.)). The material of the present invention may include a substrate that is selected from a film, woven, nonwoven, paper and laminates or combinations thereof, although any other material (e.g. steel, hard plastic, glass, etc.) or surface thereof which is suitable for printing as discussed herein is also contemplated. It is further contemplated that the material may comprise a personal care product or one or more components thereof.

In yet another alternative embodiment, the present invention is also directed to an absorbent article comprising (i) a substrate, the substrate having at least a first surface; and (ii) one or more chemistries, where at least one of the chemistries, is at least in part, a phase-change liquid; and wherein the one or more chemistries are applied to the substrate in one application so as to produce a substrate having desired topographical characteristics. As noted throughout, exemplary phase-change liquids, materials or applications include, but are not limited to, waxes, petrolatum based lotions, adhesives, thermoplastics, and the like. The application or applications contemplated by the present invention include those which are intended for topical and/or internal use. Although the desired embodiments of the present invention are directed to use with or in personal care products or the like, where the use of skin unfriendly components generally needs to be limited or avoided, in those instances where the application is used to print on something other than items or materials which will contact or be used intimately with the skin, any suitable components may be used. Thus, the only limitation on applications which may be used in connection with the present invention is that the applications must be capable of being processed by the apparatus of the present invention and being capable of adhesion to the selected substrate. The suitability of a particular substrate may depend, at least in part, on the applications or chemistries being used in conjunction therewith.

The substrate of the absorbent article will desirably be selected from a film, woven, nonwoven, paper and laminates or combinations thereof and may form, for example, a personal care product or component thereof. The one or more chemistries may be selectively applied to all or a portion of the substrate of the absorbent article, may be applied to the substrate in a pattern and/or may be applied to the substrate so as to create a topography of chemistry.

The generated pattern or patterns of application desirably, but need not, include overlapping or partially overlapping segments, and the pattern may be one which is repeating, non-repeating or random. Where a topography of chemistry is created on the substrate, the substrate will desirably exhibit skin health benefits such as improved fluid management (e.g. liquid wicking or flow management applications), and/or skin separation. Desirably, the enhanced fluid management may include, but need not be limited to, such topography or topographical properties such as pixel resolution topical features built in three dimensions, hydrophobic domes discretely placed within a field of hydrophilic substrate, hydrophobic grids or patterns surrounding hydrophilic substrate domains, printed capillary wicking gradients (which may be accurate to micrometers), and various other forms of wicking and permeability barriers. In addition, the material of the present invention may also include chemistries or applications which create nondestructive bonding of the material into stabilized webs, interfacial bonding of the substrate (when more than one substrate layer is present) to form laminates, user unzippable bonds (e.g. debondable body contour lines to allow the component or product to fit a larger body) or other software-driven graphics. In another aspect of the absorbent article of the present invention may have discrete segments having a substantially semicircular cross-section extending above the body-facing surface of the substrate. Further still the discrete segments of the absorbent article will desirably have a volume in the range of about 5 nanoliters to about 400 nanoliters. As above, the one or more chemistries contemplated by the present invention include any chemistry, application or composition or the like which is capable of adhering or being applied to a suitable substrate so as to produce the materials discussed herein, and may include, but is not limited to, medicaments, inks, waxes, paints, lotions, ointments, skin health agents, topical applications, and the like or combinations thereof. In one embodiment at least one of the chemistries used will desirably be or will desirably comprise, at least in part, a phase-change material.

One embodiment of the material of the present invention produced by the apparatus referred to below will have droplets or discrete segments, desirably of about 1-2 mm diameter each, of at least one molten (and desirably a phase-change) liquid (i.e. chemistry or application) applied thereto. Although droplets or discrete segments of particular cross-sectional shapes, dimension or volume are contemplated and preferred in certain embodiments, in those embodiments not requiring specific segment size or shape, any variety of cross-sectional shapes of the droplets or segments are contemplated for use on or in the material of the present invention. The cross-sectional shape of the droplets or segments which solidify on the surface of the substrate may be changed or controlled, at least to some degree, depending on the selection of the chemistries or applications to be applied to the selected substrate, as well as the apparatus or method selected for application. Specifically, the cross-sectional shape of the droplets which solidify on the surface of the substrate may be changed, by manipulating, for example, the temperature, velocity, and throw distance. Thus, for example, if the temperature of the liquid is increased, it will typically penetrate further into the substrate before solidifying, thereby resulting in a more dome-shaped deposit having less height than one formed at a lower temperature. The droplets may be deposited on a wettable substrate at desired X-Y intervals, thus providing, in this example, at least two desirable attributes. The first being that the surface is covered with raised, rounded, hydrophobic domains that force liquid away and into the hydrophilic surrounding field, leaving the domes clean and dry, and the second being that the domes, all being substantially the same height provide a uniform spacing between the wet substrate and the user's skin. Of course, depending on the intended function of the domes (e.g. liquid barrier, fluid management, skin separation, aesthetics, etc.), and whether the application is intended to be permanent or releasable, the desired makeup, including, for example, weight, shape and composition, of the discrete segments applied will and should carefully selected. While a vast number of skin health benefits may be achievable with the use of certain chemistries or applications, one skilled in the art will recognize and appreciate that different chemistries or compounds will work better on certain substrates or under certain conditions.

The material of the present invention may be produced by any number of suitable methods and apparatus, such as those disclosed in commonly assigned U.S. patent application Ser. No. ______, filed on Nov. 16, 2001 and entitled “APPARATUS AND METHOD TO PRODUCE TOPOGRAPHY, UNIQUE FLUID HANDLING PROPERTIES AND BONDING PROPERTIES ON AND WITHIN SUBSTRATES”, the disclosure of which is incorporated herein by reference in its entirety.

Exemplary substrates include, but are not limited to, wovens, nonwovens, paper, films, tissue, metals and generally any surface of any product which is capable of having the application or applications described herein applied thereto either in the manner described or so as to produce the materials discussed herein.

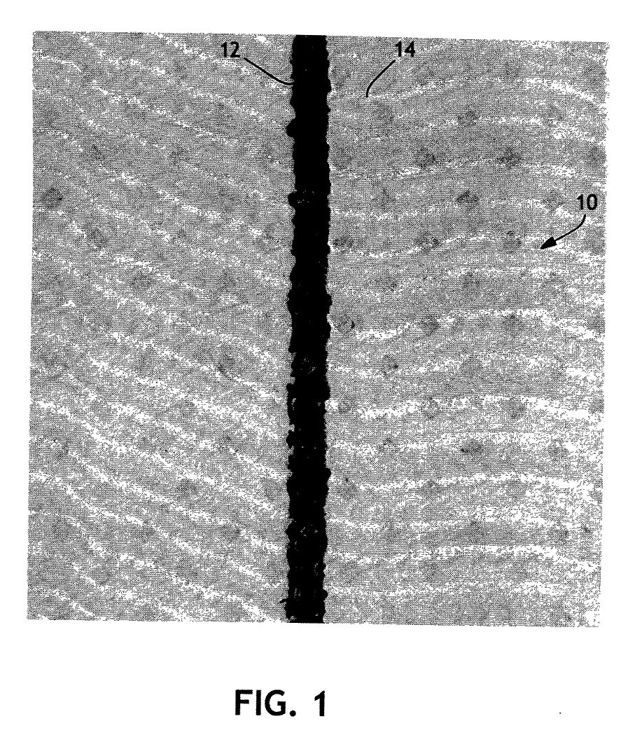

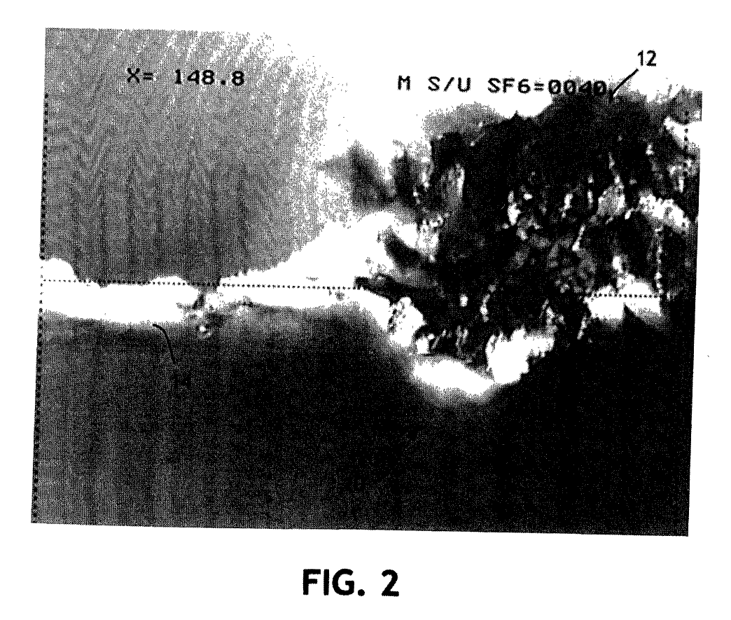

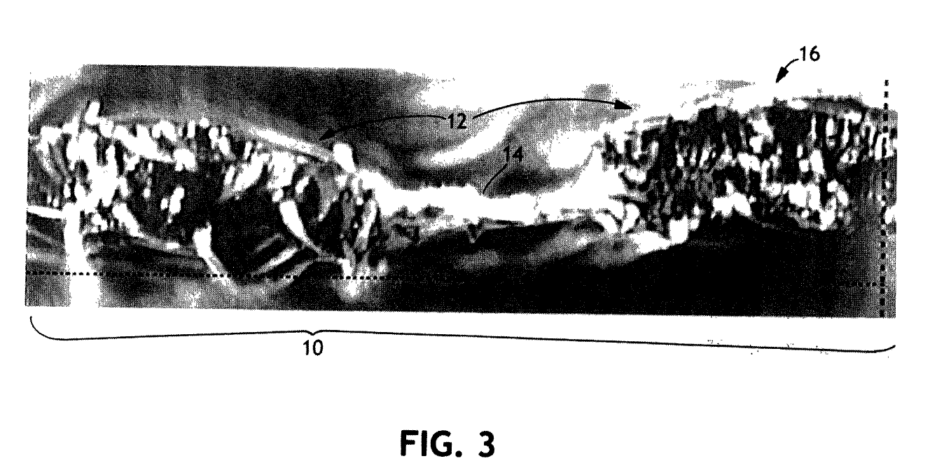

Turning to FIG. 1, there is illustrated a representative material of the present invention. The exemplary material 10 is shown in FIG. 1 with one line of application or chemistry 12 applied to the top surface of the substrate 14. As shown in FIG. 1, but more clearly illustrated in FIGS. 2 and 3, the one or more chemistries or applications 12, in this case wax, which are applied to the substrate 14, can be built up into distinct topographic features 16 by discrete placement, so as to produce a material which is much thicker than the base material. The topographic features may be used independently or may be used together as shown in FIG. 3. In FIG. 3, two neighboring lines of wax 12 have been applied to the substrate 14 such that they promote the channeling of fluid and force directional wicking along the substrate 14. One skilled in the art will appreciate that multiple variations and combinations of the applications and chemistries are possible and that each is intended to be covered by the disclosure of the application. For example, in some embodiments, it may be desirable for the material to have but one line of application to act as a simple barrier to one or more fluids and which may provide for directional wicking. Alternatively, it may be desirable for the applications to be applied as shown in FIG. 3 to control fluid movement to a specific area or component of a material or product. This can be achieved in a number of ways including, for example, varying the height, number and/or penetration and thus the topography of the chemistries applied to the substrate. In addition to the varying the number, height, and penetration of the chemistries, the chemistries are desirably selected for their physical attributes (e.g. affinities or repulsions to certain fluids) so as to produce the most desirable and efficacious end product.

While much of the disclosure contained herein is directed to the use of valve jet printing apparatus to produce the materials of the present invention, embodiments of the present invention may alternatively be executed by use of a piezo-driven printhead. The piezo-driven print devices are typically capable of emitting droplets having a diameter in the range of about 50-90 micrometers with placement resolution to about 1/200 of an inch. In this instance the micro-droplets may be deposited in a pre-described pattern wherein continuous patterns of ink enclose discrete domains of the wettable substrate. Although, the apparatus of the present invention may operate (e.g. have lengthier dwell times or have multiple rows of valves, etc.) such that the printing may be accomplished in one pass of the apparatus over the substrate or one pass of the substrate by the apparatus, in some embodiments of the material of the present invention it may be desirable for the chemistry or application, and hence the pattern, topography and/or the fluid management characteristics, to be produced or achieved by multiple passes of the substrate past the printhead. The multiple pass approach may be desirable for a number of reasons including, but not limited to, the alteration of each layer from the original in such a way that the cross-sectional shape of a pattern element is desirably developed to be, for example, triangular, or hemispherical. Additionally, it may be desirable to produce a material via multiple passes of the substrate past the printhead where releasable treatments or chemistries are used such as those disclosed, for example, in commonly assigned U.S. patent application Ser. No. 09/938,347 to Yahiaoui et al.

In another embodiment the technique of the present invention may be used to print fluid wicking or flow management devices directly onto a chosen substrate with high degrees of accuracy. For example, if it is desired to create a capillary wicking device to transport a certain liquid from a first point on or in the material to a second point on or in the material, while also increasing the width of the wicking field, the idealized material may be digitally realized using a graphics software program. The program may be constrained to use mathmatic requirements particular to the fluid, substrate, and ink such as capillary size, length, pressure, etc, to design the device. Once created, the design may be accurately produced on the substrate by printing. The use of computer generated print designs or computer operated print heads allows for nearly limitless design configurations.

Yet another embodiment of the material of the present invention involves various forms of bonding. For example, a pattern of droplets emitted from a solenoid valve printhead may be printed on a moving consolidated mat of fibers. The mat can then be compressed and heated to remelt the droplets. The droplets could flow around the fibers, and when solidified stabilize the mat into a bonded web without having damaged the fibers. Alternatively, the pattern of droplets or discrete segments may be applied to one layer of a material before another layer of material (e.g. film, web, etc.) is placed over the first. Upon compression, reheating, and cooling, an interfacial bond may be formed. Because the bond patterns are digitally generated, they are infinitely variable and instantly changeable. The use of phase change liquids or materials as discussed herein further enhances the number of possible bond patterns which may be suitable and can enable the use of certain bond patterns with certain materials or substrates which may have heretofore been unsuitable. That is, the use of phase-change applications can enable different substrate penetration or adhesion thereto than previously obtainable with non-phase-change applications. Accordingly, bonding which may have been heretofore unobtainable or which if obtainable was too weak, can in some instances be enhanced. Additionally, where the bond strength was previously sufficient, the number of bonds may be able to be reduced, thereby potentially providing cost savings and/or increased production speed.

One skilled in the art will appreciate in light of the disclosure herein that in any given product length or length of web the bond pattern may have a range of bond density, or zones of differing bond density. The droplets or discrete segments may be placed so that upon remelting they become contiguous rendering that zone of the web selectively impermeable. In addition, the droplets may be deposited in a manner that they become a reversible or “unzippable” bond line to be employed by the user to, for example, facilitate fit. For example, a disposable pant may be fabricated having unzippable bond lines that force the pant to conform closely to user being of the intended minimum weight range of the product. The unzippable bond lines may be separated by the user thus bringing the next larger bond lines to force conformation to the next larger weight with the range of the product.

While each product or component of the present invention may require different features or qualities, in at least one product contemplated by the inventors, it would be desirable to include combinations of all the embodiments listed above. For example, a region of hydrophobic spacing droplets may graduate into liquid channeling lines, and further into a micro-wicking region. Liquid channeling lines may simultaneously bond layers, graduate to discrete bond points, with further gradation in bond point density.

While the present invention has been described in connection with certain desired embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

We claim:

1. A material having fluid handling characteristics, said material comprising:
a substrate, having at least a first surface; and
one or more chemistries applied to the first surface of the substrate in discrete droplets;
wherein one or more droplets form discrete domains on the substrate and wherein the domains produce a topography of chemistries upon the substrate and wherein the domains provide enhanced fluid handling characteristics.

2. The material of claim 1, wherein said one or more chemistries and the chemistries applied to the substrate have varying degrees of penetration; and wherein the varying degrees of chemistry penetration produce materials having various topographies.

3. The material of claim 1, wherein the one or more chemistries are selectively applied to the substrate.

4. The material of claim 1, wherein the one or more chemistries are applied to the substrate so as to produce a selectively located fluid barrier.

5. The material of claim 1, wherein said one or more the chemistries are applied to the substrate in a pattern.

6. The material of claim 1, wherein the topography of chemistries results in a substrate which exhibits improved fluid management and/or skin separation.

7. The material of claim 1, wherein the discrete domains direct the wicking of fluids contacting the surface of the material.

8. The material of claim 7, wherein at least one of the chemistries is releasable, at least in part, from the substrate when exposed to temperatures at or above about 23° C.

9. The material of claim 1, wherein the substrate is selected from a film, woven, nonwoven, paper and laminates or combinations thereof.

10. The material of claim 1, wherein the material comprises a personal care product or one or more components thereof.

11. The material of claim 10, wherein the personal care product is selected from a diaper, training pant, absorbent underpant, adult incontinence product, sanitary wipe, wet wipe, feminine hygiene product, wound dressing, nursing pad, time release patch, surgical gown, fenestration drape, bandage, mortuary product, veterinary product, hygiene and absorbent product.

12. An absorbent article comprising:
a substrate, having at least a first surface; and
one or more chemistries, at least one of the chemistries being at least in part, a phase-change liquid;
wherein the one or more chemistries are applied to the substrate so as to produce a substrate having desired topographical characteristics.

13. The absorbent article of claim 12, wherein the substrate is selected from a film, woven, nonwoven, paper and laminates or combinations thereof.

14. The absorbent article of claim 12, wherein the one or more chemistries are selectively applied to the substrate.

15. The absorbent article of claim 14, wherein the chemistries are applied to the substrate in a pattern.

16. The absorbent article of claim 12, wherein the chemistry is applied to the substrate so as to create a topography of chemistry, and wherein the topography provides skin health benefits.

17. The absorbent article of claim 16, wherein the application of topography results in a substrate which exhibits improved fluid management and/or skin separation.

18. The absorbent article of claim 12, wherein the topographical characteristics include liquid wicking or flow management applications.

19. The absorbent article of claim 12, wherein the one or more chemistries are applied to the substrate so as to produce a fluid barrier.

20. The absorbent article of claim 12, wherein the substrate comprises a personal care product or component thereof.

21. The absorbent article of claim 20, wherein the personal care product is selected from a diaper, training pant, absorbent underpant, adult incontinence product, sanitary wipe, wet wipe, feminine hygiene product, wound dressing, nursing pad, time release patch, surgical gown, fenestration drape, bandage, mortuary product, veterinary product, hygiene and absorbent product.

22. The absorbent article of claim 12, wherein the discrete segments have a substantially semicircular cross-section extending above the body-facing surface of the substrate.

23. The absorbent article of claim 12, wherein the discrete segments have a volume in the range of about 5 nanoliters to about 400 nanoliters.

24. A composite comprising:
a substrate, being of at least one layer and having at least a first surface; and
one or more chemistries, at least one of the chemistries being at least in part, a phase-change liquid;
wherein the one or more chemistries are applied to the substrate so as to produce a substrate having discretely placed and registered bond points.

25. The composite of claim 24, wherein the discrete bond points are inter-fiber bond points or interfacial bond points.
